(19) United States
(12) Patent Application Publication
(10) Pub. No.: US 2015/0261936 A1
(43) Pub. Date: Sep. 17, 2015

(54) Method for Separating and Analyzing Overlapping Data Components with Variable Delays in Single Trials

(71) Applicant: Hong Kong Baptist University, Hong Kong (HK)

(72) Inventors: Changsong Zhou, Hong Kong (HK); Guang Ouyang, Hong Kong (HK); Werner Sommer, Hong Kong (HK)

(73) Assignee: Hong Kong Baptist University, Hong Kong (HK)

(21) Appl. No.: 14/210,321

(22) Filed: Mar. 13, 2014

(51) Int. Cl. G06F 19/00; A61B 5/04; A61B 5/0476

(52) U.S. Cl. CPC G06F 19/363; G06F 19/322; A61B 5/0476; A61B 5/04012

(57) ABSTRACT

The present invention relates to a method for separating and analyzing overlapping data components with variable delays in single trials. In particular, the present invention relates to a method for separating and analyzing overlapping data components consistently occur in multiple realizations but locked to different time markers with variable inter-marker delays using an extended residue iteration decomposition (RIDE) algorithm. The present invention has applications in separating and analyzing event-related brain potential (ERP) data derived from single-trial responses.

### FIELD OF INVENTION

The present invention relates to a method for separating and analyzing overlapping data components with variable delays in single trials. In particular, the present invention relates to a method for separating and analyzing overlapping data components consistently occur in multiple realizations but locked to different time markers with variable inter-marker delays using an extended Residue Iteration Decomposition (RIDE) algorithm. The present invention has applications in separating and analyzing event-related brain potential (ERP) data derived from single-trial responses.

### BACKGROUND OF INVENTION

Event-related potential (ERP) recording, widely used in Cognitive Neuroscience, provides valuable insights into cognitive brain activities. The ERP consists of several components reflecting specific perceptual, cognitive, and motor processes. In cognitive experiments the ERP is typically obtained by averaging across 10 to 100 single EEG (Electroencephalography) trials per condition, synchronized to the onsets of stimuli or responses, with the assumption that each single trial contains more or less the same sequence of sub-processes and ERP components, and the residues between the average and a given single trial are just noise (Model 1, FIG. 1A, left panel). However, in cognitive tasks, there is usually considerable trial-to-trial variability in reaction times (RT) and there are activity patterns systematically locked to stimulus onset and the variable RT, respectively (FIG. 1B), suggesting that cognitive task processing more likely follows Model 2, depicted in FIG. 1A, right panel. This model assumes a series of cognitive sub-processes and associated ERP components, which are reliably present in most single trials. The early sub-processes and components are more closely locked to stimulus onset, while of the later components, some are locked to the response, whereas others may not have any explicit time-marker and can be highly variable in latency. The conventional stimulus-locked average ERP may strongly smear and mix different components in time and space, especially late, endogenous components (FIG. 1C), and hence seriously misrepresent their time courses and topographies.

Although long recognized as a problem, the temporal variability of the EEG responses has rarely been systematically addressed. The more realistic Model 2 has not been firmly established as a new paradigm for ERP analysis although similar concepts and several corresponding ERP decomposition methods have been suggested in Hansen, J. C. (1983). Separation of overlapping waveforms having known temporal distributions. Journal of neuroscience methods 9:127-139 and Zhang, J. (1998) (the content of which is incorporated herein by reference in its entity). Decomposing stimulus and response component waveforms in ERP. Journal of neuroscience methods 80:49-64 (the content of which is incorporated herein by reference in its entity), to deal with the problems of temporal and spatial mixing of time-varying components. Previously suggested temporal decomposition methods have almost never been applied in ERP research, probably because these methods cannot deal with intermediate components without explicit time-markers and may seriously distort the components under certain circumstances, as reported in Hansen, J. C. (1983). Separation of overlapping waveforms having known temporal distributions. Journal of neuroscience methods 9:127-139 (the content of which is incorporated herein by reference in its entity) and Takeda, Y., Yamanaka, K., & Yamamoto, Y. (2008). Temporal decomposition of EEG during a simple reaction time task into stimulus-and response-locked components. NeuroImage 39:742-754 (the content of which is incorporated herein by reference in its entity). Spatial decomposition methods, for example, Independent Component Analysis (ICA) as reported by Hyvärinen, A., Oja, E., (2000), Independent component analysis: algorithms and applications. Neural Networks: 13:411-430 (the content of which is incorporated herein by reference in its entity), also aim to separate ERP into components—usually called sources—that are statistically mutual independent. ICA obtains as many independent components (ICs) as there are electrodes and it is difficult to cluster and associate all of the ICs to cognitive sub-processes and events.

In the earlier work of Ouyang, G., Herzmann, G., Zhou, C., & Sommer, W. (2011). Residue iteration decomposition (RIDE): a new method to separate ERP components on the basis of latency variability in single trials. Psychophysiology, 48:1631-1647 (the content of which is incorporated herein by reference in its entity), the earlier version of RIDE has overcome the limitation of existing ERP decomposition methods to deal with intermediate components without explicit time-markers, but there still exists several problems: 1) serious noise amplification in the signal analysis, 2) existence of separate nonsense complementary signal components, 3) severe distortions of boundaries of signal, 4) arbitrary separation of ERP trend, and 5) the reported approach was constrained in being able to separate one central component, wherein the first estimation of said central component is poor if the first template of the ERP is poor. These problems could seriously limit the application of the earlier version of RIDE in real data.

Thus, it is an objective of the present invention to provide a robust and practical method for separating and analyzing overlapping data components consistently occur in multiple realizations but locked to different time markers with variable inter-marker delay (e.g. for ERP data) with an extended RIDE algorithm that 1) overcomes all the serious limitations of its predecessor, and therefore, 2) is able to properly separate overlapping components—with or without explicit time markers—without serious distortion, 3) restores the most probable waveform of the data from the separated components, and 4) obtains the variable amplitude and latency parameters from single trials. With the present invention, EEG data can be explored more deeply to investigate brain-behavior relationships in different dimensions.

Citation or identification of any reference in this section or any other section of this application shall not be construed as an admission that such reference is available as prior art for the present application.

### SUMMARY OF INVENTION

The present invention provides a method for separating and analyzing overlapping data components with variable delays in single trials. In particular, the present invention provides a method for separating and analyzing overlapping data components consistently occur in multiple realizations but locked to different time markers with variable inter-marker delays using an extended Residue Iteration Decomposition (RIDE) algorithm.

In a first aspect of the present invention there is provided a method for separating and analyzing overlapping data components with variable delays in single trials comprising:
a) an initial latency estimation module to estimate the latency of an one or more unknown time-marker components of an one or more data components by a first template matching operation;
b) a first iterative module to decompose the one or more data components by a minimization operation based on a known or the initially estimated latency of the one or more unknown time-marker components;
c) a second iterative module wherein:
   to further estimate the latency of the one or more decomposed data components without time-markers from the first iterative module wherein the latency is estimated by a second template matching operation between the one or more data component and single trials after removal of all other data components and the further estimated latency is applied to the first iterative module to further decompose the one or more data components;
   to apply a de-trend module to remove the trend noise in the decomposed one or more data components to prevent distortion in all the iterations other than the final iteration; and
   to apply a windowing module to refine the one or more data components using window functions in all the iterations other than the final iteration;
d) an iteration termination module to terminate the iteration between the second and the first iterative modules;
e) a baseline adjustment module to adjust the baseline of the separated one or more data components from the final iteration of the second iterative module;
f) a reconstruction module to reconstruct the most probable representation of the added-up data components by summation of each separated component at their most probable latency across single trials;

In a first embodiment of the first aspect of the present invention there is provided the initial latency estimation module comprising signal processing techniques such as Woody's method.

In a second embodiment of the first aspect of the present invention there is provided the initial latency estimation module comprising signal processing techniques such as peak-picking.

In a third embodiment of the first aspect of the present invention there is provided the initial latency estimation module comprising signal processing techniques such as likelihood method.

In a fourth embodiment of the first aspect of the present invention there is provided the initial latency estimation module comprising signal processing techniques such as template matching using pre-defined templates.

In a fifth embodiment of the first aspect of the present invention there is provided the minimization operation comprising a Ln-norm operation, preferably a L1-norm operation.

In a sixth embodiment of the first aspect of the present invention there is provided the second template matching operation comprising a peak lag detection from cross-correlation between one data component and single trials wherein all other data components are removed.

In a seventh embodiment of the first aspect of the present invention there is provided the iteration termination module comprising an operation to terminate the iteration which further comprising a constraint of the estimated latency of the one or more time-markers-unknown components for each single trial to be monotonic.

In an eight embodiment of the first aspect of the present invention there is provided the reconstruction module comprising an operation to reconstruct the most probable added-up data components by summation of all the decomposed data components respectively being located at their most probable latency across single trials.

In a ninth embodiment of the first aspect of the present invention there is provided a module to provide estimates of the latency and amplitude information of the one or more data components in each single trial.

In a tenth embodiment of the first aspect of the present invention there is provided the reconstruction module comprising operations to obtain the waveforms for the one or more data components and the topographies at each time-marker of the one or more data components.

In an eleventh embodiment of the first aspect of the present invention there is provided a module to separate more than one data components with unknown time-marker components, which further comprising the application of the initial latency estimation module in different time windows.

In a twelfth embodiment of the first aspect of the present invention there is provided the one or more data components are event-related potential recordings wherein the event-related potential recordings are recordings of brain activities.

In a thirteenth embodiment of the first aspect of the present invention there is provided the one or more data components are electroencephalography signal recordings wherein the electroencephalography signal recordings are recordings of brain activities.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows two models of stimulus processing underlying ERP analysis, wherein A shows the assumed models of cognitive tasks processing; B shows the real behavior and EEG data of cognitive tasks processing; and C shows the illustration of ERP decomposition.

FIG. 2 shows the comparison of the performance of Fourier decomposition and RIDE.

FIG. 3 shows the comparison of RIDE and ICA in the simulation data.

FIG. 4 shows an example of the separation of ERPs by RIDE and the smearing effects in conventional stimulus-locked average ERP when compared to the reconstructed ERP by RIDE.

FIG. 5 shows the RIDE components for a face-recognition (A) and a lexical decision task (B).

FIG. 6 shows the latency and amplitude variability of RIDE component clusters across single trials for one participant from the primed-unfamiliar condition of the face recognition experiment.

FIG. 7 shows the trial-to-trial reliability (the selected channel was PO4).

FIG. 8 shows the comparison between RIDE and ICA in real data from a single right-handed subject of the face recognition task.

FIG. 9 shows the flow chart of the RIDE processing of EEG data and the outcomes of RIDE.

### DETAILED DESCRIPTION OF INVENTION

The present invention is not to be limited in scope by any of the specific embodiments described herein. The following embodiments are presented for exemplification only.

Herein presented is a new method, termed extended Residue Iteration Decomposition (RIDE), for establishing ERP analysis according to ERP Model 2. Based on a previous version of RIDE from Ouyang, G., Herzmann, G., Zhou, C., & Sommer, W. (2011). Residue iteration decomposition (RIDE): a new method to separate ERP components on the basis of latency variability in single trials. Psychophysiology, 48:1631-1647 (the content of which is incorporated herein by reference in its entirety), this extended version overcomes the aforementioned limitations of existing ERP decomposition methods. The present invention is able to properly separate and restore ERP components that are reliably present across trials with or without explicit time markers. And it can obtain the variable amplitude and latency parameters from single trials. With the new ERP paradigm established by the present invention, EEG data can be explored more deeply to investigate brain-behavior relationships in different dimensions.

The present invention provides a method for separating and analyzing stimulus-driven and response-related data components in single trials. In particular, the present invention provides a method for separating and analyzing overlapping data components consistently occur in multiple realizations but locked to different time markers with variable inter-marker delays using an extended Residue Iteration Decomposition (RIDE) algorithm. In one embodiment of the present invention, there is provided a method for separating and analyzing event-related brain potential (ERP) data derived from single-trial responses.

In the embodiments of the present invention, an ERP model is formulated specifying the temporal superposition of latency-variable components and elucidating the smearing effects in conventionally averaged ERPs. Then presented is the conceptual background and algorithm of the extended RIDE method and its comparison with existing temporal and spatial decomposition methods. The comparison will be done by applications of each method in both simulated and real data.

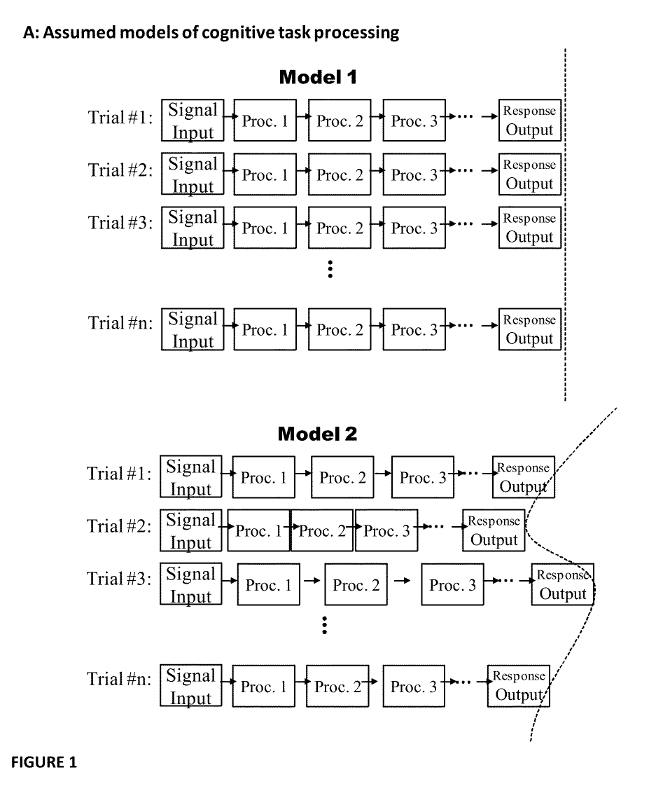

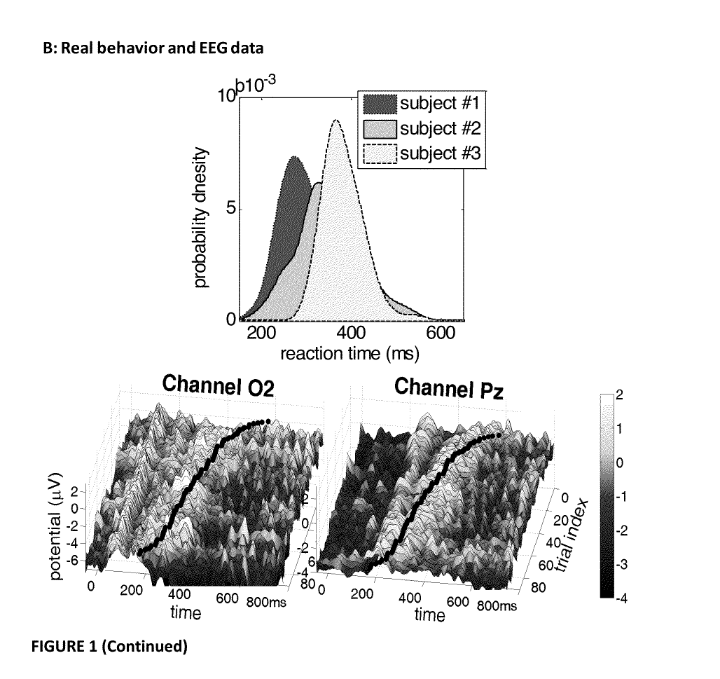

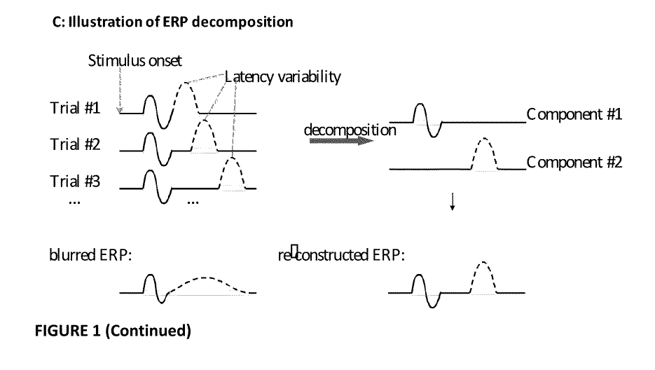

In the first embodiment of the present invention, provided are two models of the cognitive tasks processing. FIG. 1 presents two models of stimulus processing underlying ERP analysis. Stimulus is assumed to proceed through a number of processes stages or sub-processes. FIG. 1A: Model 1: Given identical stimulus conditions from trial to trial, the timing of each sequential sub-process is identical in every trial, thus stimulus-locked averaging is supposed to isolate the ERP by eliminating the noise. Model 2: The time required for each sub-process varies more or less from trial to trial even for identical stimuli. In this case, the stimulus-locked average mixes and smears the latency variable ERP components. FIG. 1B: Variability in reaction time (RT) and component latency in real experiments are consistent with Model 2. Up: The distribution of RTs of three different subjects. Down: The epochs (from −50 to 800 ms after stimulus onset) of EEG data (amplitude normalized) of one subject, sorted by RT (white dashed line). FIG. 1C: Schematic illustration of temporal decomposition of two ERP components. Left: Each single trial consists of two components, being locked to different events (e.g. to stimulus and response, respectively). The standard stimulus-locked ERP (bottom) blurs the RT-locked component and may distort the stimulus-locked component if there is overlap between both. Right: Temporal decomposition separates different components with different latency-locking. The reconstructed ERP (right bottom) is the summation of different components at their most probable latency. The smearing issue may involve more than two ERP components as shown here for illustration purposes.

### METHODS

### I. An ERP Model With Latency-Variable Components

In ERP Model 2 presented in FIG. 1A, it is assumed that each component associated with different sub-processes is reliably present in every single trial waveform but at latencies that may differ from trial to trial. There may also be trial-by-trial variability in component amplitudes. For simplicity, however, it is assumed for the moment that amplitudes are constant across all single trials. Then single trial ERPs can be described as a linear superposition of several latency-variable components:

EEGᵢ(t)=C₁(t−τ₁ᵢ)+C₂(t−τ₂ᵢ)+ . . . +Cₙ(t−τₙᵢ)+ξᵢ(t) (1)

where EEGᵢ(t) denotes the ith single trial of EEG data, Cₙ(t) the waveform of the nth ERP component, τₙᵢ the latency of the nth component for the ith trial, t the time coordinate relative to stimulus onset, and ξ the noise. It is important to stress that Eq. (1) is only a model of the complex dynamic activity in real brain activity but with a suitable method such as RIDE, this model allows us to obtain the brain activity components associated with typical cognitive sub-processes and events known to occur within certain time windows. Examples are the P1 or N1 components in visual stimulus processing, P300 in cognitive experiments, and N400 and P600 in stimulus evaluation and context updating, and N400 and P600 in language processing. Nevertheless, these components do not occur exactly at the same time across trials and are also modulated by different experimental conditions. In a typical cognitive task, the early P1 and N1 components may be rather strictly locked to stimulus onset, and the final components, reflecting motor programming and executions, may be locked to the response time; however, the latency of the intermediate components, related to central cognitive processes, may not be explicitly known. Since the latency of each component is not necessarily locked to the stimulus onset the average ERP is actually the convolution of the components with the distributions of their latencies across single trials:

ERP(t)=C₁(t)*ρ(τ₁)+C₂(t)*ρ(τ₂)+ . . . +Cₙ(t)*ρ(τₙ). (2)

Here, it is assumed a sufficiently large number of trials so that the noise term can be omitted for simplicity. ρ denotes the probability density function of the latency distribution and * is the convolution f*g=∫_{-∞}^{∞}f(t−τ)g(t)dτ. As long as the distribution is not a delta function, the convolution smears the waveform and diminishes the amplitude, blurring the representations of components in the average ERP. For the early components that show little variability the smearing effect may be negligible; but the later—cognitively determined—components may vary strongly in latency and hence would be seriously smeared and mixed with other overlapping components in the average (FIG. 1B). Such smearing and mixing not only deforms the waveform of the ERP, but also distorts the topographies, and may make it difficult or even impossible to examine experimental effects in terms of topography and amplitude, leading to misinterpretations of brain-behavior relationships. If the time course of each component Cₙ(t) and the latencies τₙᵢ from the single trials can be obtained, the ERP can be more properly represented by the summation of latency-corrected components:

ERP_{lc(t)}=C₁(t)*δ(t−τ₁ₚ)+C₂(t)*δ(t−τ₂ₚ)+Cₙ(t)*δ(t−τₙₚ), (3)

where ERP_lc is the latency-corrected ERP, δ is the delta function and τₙₚ is the most probable latency of the nth component across all single trials. If we assume that Cₙ(t) represents the waveform located at the most probable latency, Eq. (3) can be simplified as:

ERP_{lc(t)}=C₁(t)+C₂(t)+ . . . +Cₙ(t). (4)

The difference between Eq. (2) and Eq. (4) is illustrated by the two schematic waveforms at the bottom of FIG. 1C. The amplitude of the second, late ERP component will be significantly enhanced after reconstructing from latency variability.

The reconstructed ERPs in Eq. (4) from the latency-corrected components can be used like conventionally averaged ERPs to study experimental effects in terms of topographies and amplitudes of the brain activity. Moreover, the RIDE method based on the new ERP model in Eq. (1) also provides the option to study experimental effects on the waveforms and topography of each separated component, and to consider the mean and variance of the latencies and their relationship with RTs. Consequently, the scope of EEG analysis can be greatly expanded with this new approach, considering latency-variable ERP components.

According to Eq. (1), two temporally separated components with time-locked latencies without significant variability of the inter-component interval would be grouped as a single component cluster in the temporal evolution of the brain activity (e.g., P1 and N170 components). Therefore, a component cluster could contain temporally and topographically distinct components that are temporally coupled to the same physical or cognitive event. Hence, in Model 2, each sub-process may be associated to a cluster of components with latencies locked to each other. Thus, the term “component cluster” in the applications of RIDE is used.

Similar models as in Eq. (1) but considering only stimulus onsets and reaction times as time markers have been proposed previously and temporal decomposition methods have been suggested to obtain the components in the reports of Hansen, J. C. (1983). Separation of overlapping waveforms having known temporal distributions. Journal of neuroscience methods 9:127-139 (the content of which is incorporated herein by reference in its entirety), Takeda, Y., Yamanaka, K., & Yamamoto, Y. (2008). Temporal decomposition of EEG during a simple reaction time task into stimulus-and response-locked components. NeuroImage 39:742-754 (the content of which is incorporated herein by reference in its entirety), and Zhang, J. (1998). Decomposing stimulus and response component waveforms in ERP. Journal of neuroscience methods 80:49-64 (the content of which is incorporated herein by reference in its entirety). However, these previous attempts to establish an alternative ERP model have not been well accepted and hardly been applied in the field of ERP research. The problems of these models and methods are discussed below.

### II. Temporal Decomposition Methods

The objective of temporal ERP decomposition is to solve the time courses of each component Cₙ(t), given that the latencies τₙᵢ are all known. Several methods for temporal decomposition have been suggested for ERPs, such as (1) Fourier decomposition by Hansen, J. C. (1983). Separation of overlapping waveforms having known temporal distributions. Journal of neuroscience methods 9:127-139 (the content of which is incorporated herein by reference in its entirety), and Takeda, Y., Yamanaka, K., & Yamamoto, Y. (2008). Temporal decomposition of EEG during a simple reaction time task into stimulus-and response-locked components. NeuroImage 39:742-754 (the content of which is incorporated herein by reference in its entirety); 2) iterative deconvolution by Zhang, J. (1998). Decomposing stimulus and response component waveforms in ERP. Journal of neuroscience methods 80:49-64 (the content of which is incorporated herein by reference in its entirety); and 3) General Linear Model (GLM) decomposition by Dandekar, S., Privitera, C., Carney, T., & Klein, S. A. (2012). Neural saccadic response estimation during natural viewing. Journal of neurophysiology, 107:1776-1790 (the content of which is incorporated herein by reference in its entirety). In fact, these methods are mathematically equivalent. Unfortunately, previously suggested temporal decomposition methods have not received much attention in the ERP literature. Most citations of temporal decomposition methods have been on the conceptual level. One reason for this relative neglect may be the assumption of these methods of exclusively marker-locked (e.g., stimulus- or response-locked) components; hence they cannot deal with intermediate components without explicit time markers, such as the cognition-related components P3, N400, P600, etc. In addition, these methods cannot be applied when there is no response. As an exception in Takeda, Y., Sato, M. A., Yamanaka, K., Nozaki, D., & Yamamoto, Y. (2010). A generalized method to estimate waveforms common across trials from EEGs. NeuroImage, 51:629-641 (the content of which is incorporated herein by reference in its entirety), it was proposed to extract ERP components without explicit time markers by random search of the latency to minimize the residues, a method suffering from the problem of local minima. Apart from the shortcomings in psychological aspects, it is shown below that these temporal decomposition methods have an inherent divergence issue in the case of low latency variability, which can strongly distort the separated components in the presence of noise. These problems greatly limit the applicability of these methods in ERP analysis and may be a main reason why ERP analysis has remained to be restricted to the conventional average implicitly accepting the model of latency-invariant ERP components despite of these promising previous attempts.

Given that all the latencies are known for Eq. (1), a standard expression of the theoretical solution of the components in (1) can be found. Basically the solution is derived from the general linear model using the least square estimation is:

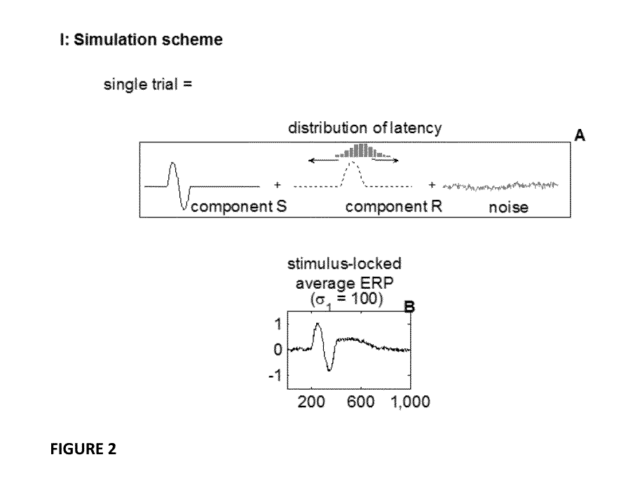

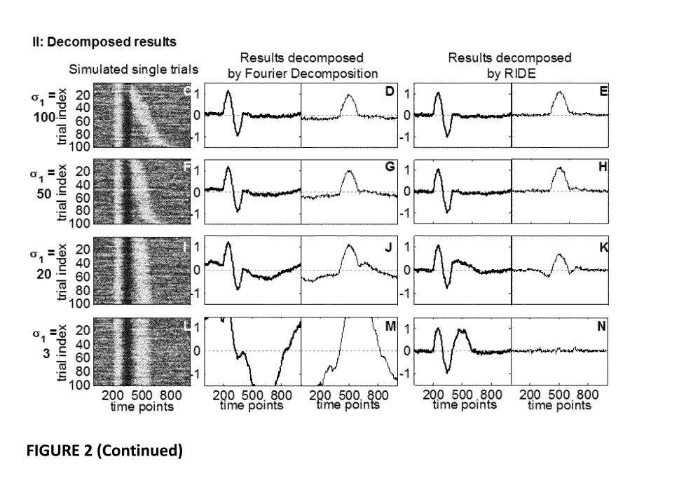

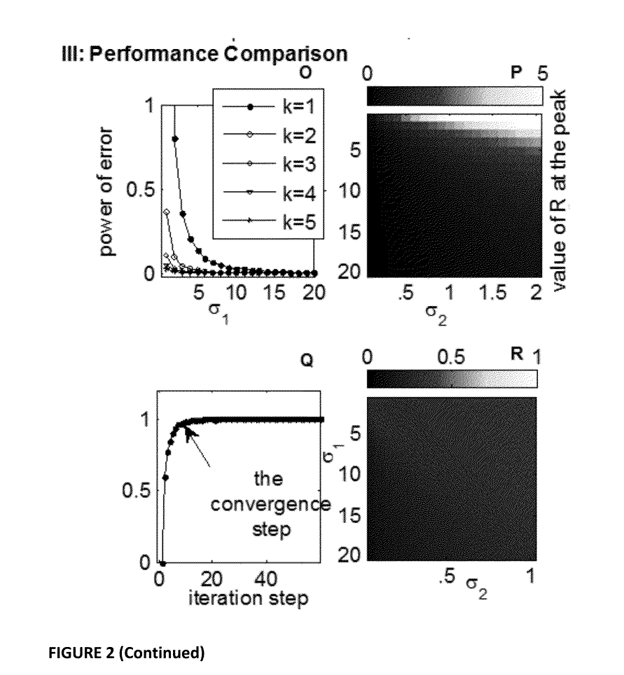

C(k)=(L^{T}(k)L(k))⁻¹L^{T}(k)EEG(k) (5)

where EEG(k) is the signal matrix, C is the component matrix, L is the coefficient matrix containing the latencies of each component and k is the frequency. Every term in (5) is the representation in the Fourier domain. The least square estimation in Eq. (5) gives poor results when the covariance matrix L^{T}(k)L(k) is close to singularity, i.e., when the latencies of two of the components are effectively locked to each other and do not have clear variability from each other. In this circumstance, the components are locked to each other in latency and should be regarded as a single component cluster rather than two different ones with separate time markers. Otherwise the least square solution in Eq. (5) would give rise to two complementary waveforms, which makes no psychological sense (see simulation results in FIG. 2M). In general, due to the intrinsic noise, the least square estimation has certain deviation from the actual component:

Cₙ′(k)=Cₙ(k)+ε(k). (6)

Here Eq. (6) is again in Fourier domain and ε(k) is the error term as a function of the frequency k. Considering a simple model with two components S and R, It can be shown (see Appendix) that the magnitude of the error term has the following frequency-dependent relationship with the variability (standard deviation σ) of the latency difference between the two components:

$$\vert ɛ ( k ) \vert ∼ \frac{1}{σ {k}^{2}} \qquad ( 7 )$$

The error term (7) will diverge and the noise will induce a serious distortion when the latency jitter between the two components is too small. The distortion is especially serious in the low frequency range, which explains the low frequency expansion observed in some reports such as Hansen, J. C. (1983). Separation of overlapping waveforms having known temporal distributions. Journal of neuroscience methods 9:127-139 (the content of which is incorporated herein by reference in its entirety), and Takeda, Y., Yamanaka, K., & Yamamoto, Y. (2008). Temporal decomposition of EEG during a simple reaction time task into stimulus-and response-locked components. NeuroImage 39:742-754 (the content of which is incorporated herein by reference in its entirety) about temporal ERP decomposition methods. For the decomposition of two components (S and R), the distortion occurring in both S and R yields opposite and complementary output waveforms, which can be witnessed in previous works such as Hansen, J. C. (1983). Separation of overlapping waveforms having known temporal distributions. Journal of neuroscience methods 9:127-139 (the content of which is incorporated herein by reference in its entirety), though the problem has never been formally addressed.

The divergence problem holds for all previously suggested temporal decomposition methods due to their common mathematical foundation. Mathematically, the divergent results are indeed the solution that minimizes the square error (L2 norm minimization). However, they are psychologically unacceptable because the mathematical result indicates that the ERP with normal amplitude is constituted by sub-components with large but opposite amplitudes when their latencies are too tightly locked. In the presence of strong noise, the error term (7) strongly contributes to the separated components and distorts them when latency variability is not strong enough; thus traditional temporal decomposition methods may be unable to properly separate ERP components in real, highly noisy data. To prevent divergence and distortion, a L1 norm minimization-based RIDE is proposed and described below. The systematic model simulations in the results section will show that it can avoid the distortion for small latency variability and strong noise. As shown below, in real data, L1 norm-based RIDE indeed shows much stronger consistency of separated components across participants and experiments as compared to the L2 norm-based RIDE version by our earlier work in Ouyang, G., Herzmann, G., Zhou, C., & Sommer, W. (2011). Residue iteration decomposition (RIDE): a new method to separate ERP components on the basis of latency variability in single trials. Psychophysiology, 48:1631-1647 (the content of which is incorporated herein by reference in its entirety).

### III. Residue Iteration Decomposition (RIDE)

RIDE was initially proposed in Ouyang, G., Herzmann, G., Zhou, C., & Sommer, W. (2011). Residue iteration decomposition (RIDE): a new method to separate ERP components on the basis of latency variability in single trials. Psychophysiology, 48:1631-1647 (the content of which is incorporated herein by reference in its entirety) to solve the problem of separating ERP components without time markers with an iteration procedure; however, in some datasets it produced inconsistent results. These inconsistencies are rooted in the distortion problems due to L2-norm minimization similar to previous temporal decomposition methods. They can be solved elegantly with a simple, but efficient scheme of introducing L1-norm minimization into RIDE: replacing mean waveform by median waveform during the iteration. The algorithm is outlined below.

a) Assumptions of RIDE

A main novelty of RIDE is the assumption of component constitution following the general Model 2 in Eq. (1). RIDE does not only separate components with explicit time markers, though time markers still serve as triggers for deriving marker-locked components. In typical tasks with an overt responses, one may assume at least three component clusters in single trial ERPs, the stimulus-locked component cluster S, the response-locked component cluster R and an intermediate component cluster C without explicit time marker. A component cluster may contain several components (e.g., P1 and N1), which are all locked to the same time marker. The component cluster C is neither locked to stimulus onset nor to the response, but has a significant latency jitter and is detected by template matching (see procedure below). The separation of component cluster C from S and R is based on the idea that some components might not be locked to explicit external time-markers but still consistently exist in the ERP and may represent important cognitive processes such as decision-making and memory encoding and retrieval, situated between stimulus processing and response preparation and execution. Another advantage of introducing C is that it solves the problem that in many situations a response is not be available as time-marker, for example in Nogo experiments, counting and reading tasks. In principle, there could be even more than one C component cluster, which will be discussed below. In RIDE, the latency of these components will be estimated and then updated iteratively.

b) Procedures

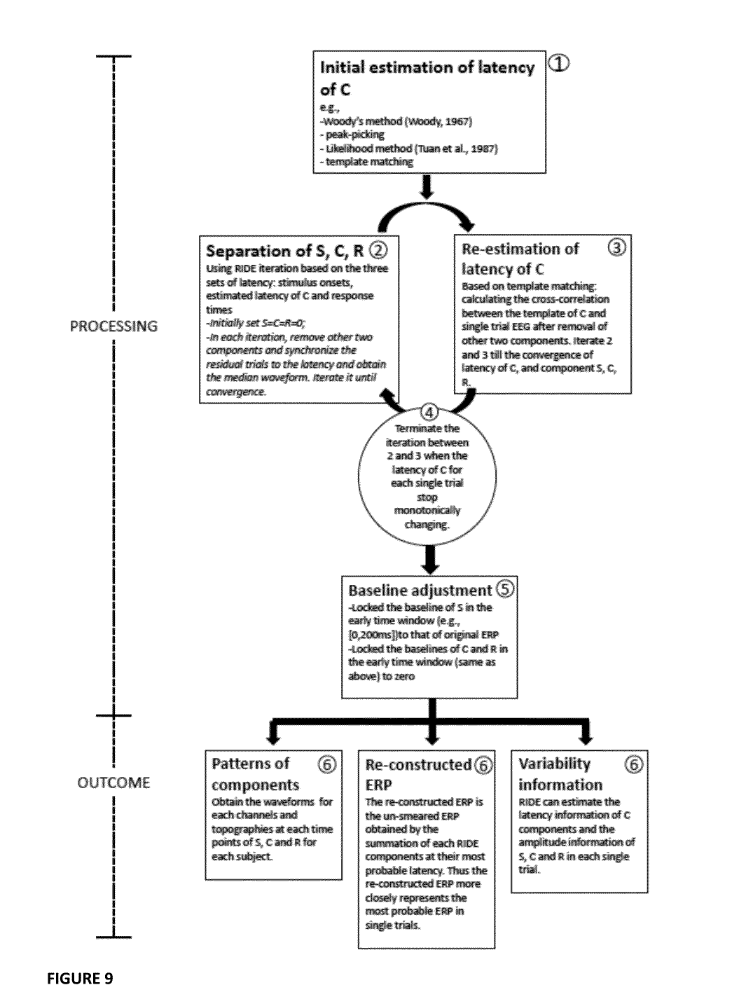

RIDE consists of a decomposition module as the inner iteration loop and a latency estimation module as the outer iteration loop forming nested iterations (see flow chart in FIG. 9). The procedure is elucidated below using the example of separating S, C, and R (assuming one C component cluster only). It can be adjusted to other cases, e.g., no R component, or more than one C components.

Decomposition Module.

Given the time-markers, RIDE decomposes ERP components in an iterative way, applying L1 norm minimization to prevent distortion. Consider the example of three component clusters S, C, and R corresponding to stimulus-locked, intermediate and response-locked components with latencies L_{S}, L_{C} and L_{R} respectively (here assuming they are all known already, recorded for S and R and estimated for C), and initially set S(t)=C(t)=R(t)=0. In each step RIDE estimates S, C, and R. To estimate S, RIDE subtracts C and R from each single trial, aligns the residual trials to the latency L_{S} in order to obtain S as the median waveform over all time points. The same procedure is applied to obtain C and R. The whole procedure is iterated to improve the estimation of the components till convergence. The convergence is effectively defined as the difference of the values of subsequent iterations is greatly smaller (here is <10⁻³) than that of the initial two iterations. In real data, the decomposition is conducted for each single electrode channel separately, but using the same time markers for all electrodes, allowing to obtain the topographies of the separated components.

Unlike the first version of RIDE presented in Ouyang, G., Herzmann, G., Zhou, C., & Sommer, W. (2011). Residue iteration decomposition (RIDE): a new method to separate ERP components on the basis of latency variability in single trials. Psychophysiology, 48:1631-1647 (the content of which is incorporated herein by reference in its entirety), in the present algorithm the median waveform was obtained after aligning the residual trials. The application of median waveform has a strong impact on the performance and stability of the method. Using median waveforms during the iteration prevents the distortion problem encountered by many temporal decomposition methods. Basically, using the median waveform minimizes the L1 norm Σᵢ₌₁ⁿ|xᵢ| of the residual error which is rather robust against outliers in noisy data, whereas the mean waveform in the first version minimizes the L2 norm Σᵢ₌₁ⁿxᵢ² as the least square estimation in Eq. (5), which is sensitive to outliers and skewness of the fluctuation distribution. The effect of median waveforms in preventing the distortion will be demonstrated in the result section (FIG. 2). In FIG. 2, it is shown the comparison of the performance of Fourier decomposition (which is based on L2-norm minimization) and RIDE.

Latency Estimation Module.

When considering the model with component clusters S, C and R, it is assumed that S and R are stimulus- and response-locked, respectively. In contrast, component cluster C is assumed to be located in between without explicit latency information; hence, a latency estimation procedure is required for L_{C}. RIDE uses a self-optimized iteration scheme for latency estimation, starting with an approximate initial estimation of L_{C} from the raw data. The initial estimation of L_{C} can be derived, for example, by peak-picking from low-pass single trials, Woody's method as reported in Woody, C. D. (1967). Characterization of an adaptive filter for the analysis of variable latency neuroelectric signals. Medical and biological engineering, 5: 539-554 (the content of which is incorporated herein by reference in its entirety), or a likelihood method as suggested in Tuan, P. D., Möcks, J., Köhler, W., & Gasser, T. (1987). Variable latencies of noisy signals: estimation and testing in brain potential data. Biometrika, 74:525-533 (the content of which is incorporated herein by reference in its entirety). Specifically, Woody's method uses the average ERP within a certain time window or a half sinusoidal function as a template to estimate the single trial latency by cross-correlation. Here, Woody's method is extended to the whole electrode set: cross-correlation time courses are calculated for each single electrode and are averaged across all electrodes. The lag of the maximum in the averaged cross-correlation time course is taken as the single trial latency (L_{C}). Starting with this L_{C} estimate, the analysis is subjected to the following iteration: 1) Use L_{S}, L_{C} and L_{R} to decompose S, C and R using the Decomposition Module till convergence. 2) Remove S and R from each single trial and calculate the cross-correlations between the residue and C component. The time point with the highest cross-correlation (averaged across scalp) is used as new estimate for L_{C}. 3) Return to 1) and 2) and iterate until convergence of both the latency L_{C} and the component clusters S, C and R. The evolution of the latency estimates for each single trial during the iterations is constrained to be monotonic, i.e., the iteration is terminated once the estimated latency value in the present iteration changes direction.

RIDE is not restricted to three component clusters S, C and R. In fact, in some experiments there is no response—not allowing for an R-component cluster—and for some situations one may consider more than one C component cluster more or less independent of each other in their latency variability; e.g., during language processing there may be N400 and P600 components that are not strictly time-locked to each other. In response-free data, the steps for estimating R can be dismissed in the Decomposition Module. For the case of separating more than one C component cluster, the initial latency of each component can be estimated within different pre-specified time window for different C component clusters (e.g., 300-500 ms for N400 and 500-800 ms for P600 in language processing, etc.).

The ERP components are not supposed to occur anywhere along the time axis, e.g., before stimulus onsets or far after response time. Therefore, the extraction of each component cluster during the iterations is confined within a specified time window where the components are assumed to occur. The confinement is implemented by applying window functions in each single trial on the pre-specified time window before each component is extracted by obtaining the median waveform of each single trial after removal of all of other components. Specifically, the time window function is multiplied with the single trials point to point within the pre-specified time window. The time window function can be, for example, Tukey window of Harris, F. J. (1978). On the use of windows for harmonic analysis with the discrete Fourier transform. Proceedings of the IEEE, 66:51-83 (the content of which is incorporated herein by reference in its entirety), wherein

$$w ( n ) = \{ \begin{matrix}0.5 ( 1 + cos ( π ( \frac{2 n}{α ( N - 1 )} - 1 ) ) & 0 \leq n \leq \frac{α ( N - 1 )}{2} \\ 1 & \frac{α ( N - 1 )}{2} \leq n \leq ( N - 1 ) ( 1 - \frac{α}{2} ) \\ 0.5 ( 1 + cos ( π ( \frac{2 n}{α ( N - 1 )} - \frac{2}{α} - 1 ) ) & ( N - 1 ) ( 1 - \frac{α}{2} ) \leq n \leq ( N - 1 )\end{matrix}$$

where n is the index (from 0 to N−1), N is the length of the time window and α is the parameter describing the sharpness of the boundary. α is adjustable. This window function was used in the present applications and 0.4 was chosen for α. The application of Tukey window can effectively prevent the boundary distortion of components. In data with poor quality, especially with high drifting artifacts, the de-trend techniques can be applied to each single trial on the corresponding time window before the application of Tukey window. The de-trend technique is implemented by subtracting the trend determined by the mean values of left margin and right margin of the time window (e.g., ⅕ of the epoch was used in the real data in present applications). The applications of de-trend and Tukey window are withdrawn in the last iteration to guarantee that the summation of the stimulus-locked RIDE components is equal to stimulus-locked average ERP. De-trend and Tukey window were not used in simulation data in the demonstration below.

Additional Technical Procedures

Baseline Adjustment.

No decomposition method is able to separate the constant term into different components with a well-defined mathematical reason. Therefore, all of the decomposition methods require pre-mean of the original data. After the decomposition by RIDE, each component is re-baselined. The strategy of re-baselining of each component is to adjust the baseline of component S to have the same baseline value of conventional average ERP in the early time window, e.g., [0, 200 ms], and to set the components C and R to have zero baseline values in [0, 200 ms]. This scheme of adjustment is based on the plausible assumption that waveform in the early period should be in line with the original ERP for the stimulus-locked component cluster S and should be zero (i.e., no activation) for the later component clusters (C, R), since they are not yet activated.

Low-Pass Filter in the Estimation of C Latency.

The cross-correlation between template and single trials to estimate the latency of C in single trial are used. Due to the intrinsic oscillatory noise of EEG data, esp., alpha wave, the peak of cross-correlation curve is easily to be distracted by high frequency noise and as a result the high frequency component will be absorbed to C component cluster along the iterations. So a low-pass filter (3 Hz-5 Hz is found to be optimal in generic cases) to the cross-correlation is employed in RIDE.

Template for Initial Estimation of C Latency.

Although epoch of stimulus-locked ERP can be used for initial estimation of latency of C, when the ERP is too strongly blurred, this might not be a good option. Half sinusoidal function was used to be suggested as a template. It is found that Hanning window performs very well as a template and is more reasonable because it reflects a continuous emergence of ERP components.

### IV. Comparison of RIDE and ICA

Since RIDE is a method for separating the ERP into different component clusters it is worthwhile comparing it to other component separation methods. One of the most popular component separation methods is Independent Component Analysis. Whereas RIDE aims to separate ERPs into components that are temporally locked to different events, ICA aims to separate independent source components that have different spatial weights and fluctuate independently in time. One may wonder if ICA is also suitable to perform the component separation defined in Eq. (1) and to associate the components to cognitive sub-processes and events of interest.

Unlike temporal decomposition, ICA requires recording of the signal from multiple electrodes to separate independent source components with different spatial distributions across the scalp. ICA is useful in the efficient removal of obvious and strong artifacts (e.g. eye blinks) from ERP. However, as the ERP components associated with cognitive processes are much weaker and subtle and might be temporal (highly) correlated, the applicability of the ICA may be limited: sources with highly-correlated activity but different scalp distribution or sources with un-correlated activity but similar distribution would not be separable by ICA. Moreover, it remains an issue in ICA to interpret and cluster the multiple ICs. RIDE and ICA are compared both in simulated and real data.

ICA was implemented using the runica function in EEGLAB toolbox by Delorme, A., & Makeig, S. (2004). EEGLAB: an open source toolbox for analysis of single-trial EEG dynamics including independent component analysis. Journal of neuroscience methods, 134:9-21 (the content of which is incorporated herein by reference in its entirety). The comparison shows that RIDE and ICA have different applicability in the decomposition of temporal and spatial components and RIDE would be especially suitable to establish the new Model 2 of ERP analysis for latency variable components.

### V. Simulation Model and Experimental Data

Simulation Data

In a first step, RIDE is compared with existing temporal and spatial decomposition methods with simulation data of ERP Model 2. For a clear demonstration, the simplest case of two components, one stimulus-locked and other response-locked is assumed. According to Model 2 in Eq. (1) (FIG. 1A), the simulated single trial ERP was composed of components S and R and pink noise (with 1/f frequency spectrum), as schematically illustrated in FIG. 2A. Each trial consisted of an S component (sine wave with amplitude 1.0) with constant waveform and fixed latency (i.e., stimulus-locked), and an R component (half sine wave with amplitude 1.0) with constant waveform but Gaussian distributed latency τᵢ with standard deviation σ₁ across trials, and pink noise with standard derivation σ₂. The latencies of R represent the reaction times. The epoch length of each trial was 1000 dots and the trial number was 100. The performance of RIDE was compared with previous temporal decomposition methods such as Fourier decomposition, Hansen, J. C. (1983). Separation of overlapping waveforms having known temporal distributions. Journal of neuroscience methods 9:127-139 (the content of which is incorporated herein by reference in its entirety); and Takeda, Y., Yamanaka, K., & Yamamoto, Y. (2008). Temporal decomposition of EEG during a simple reaction time task into stimulus-and response-locked components. NeuroImage 39:742-754 (the content of which is incorporated herein by reference in its entirety) in terms of their robustness against divergence and distortion in a broad parameter region of latency variability σ₁ and noise strength derivation σ₂ (FIG. 2). Here, a single EEG channel is considered only.

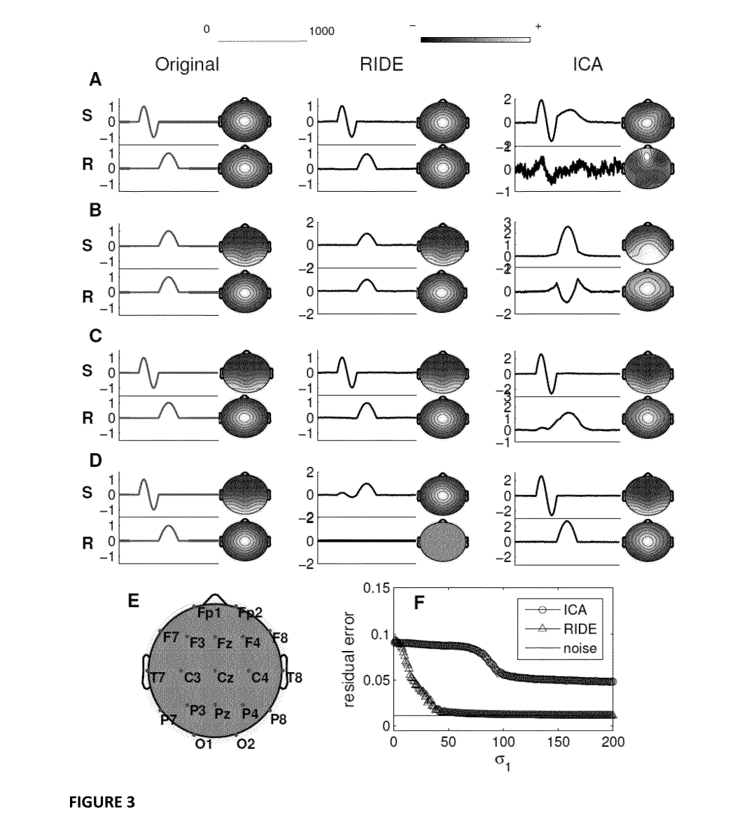

For comparisons between RIDE and ICA, EEG data is simulated according to Model 2 in Eq. (1) with 19 channels uniformly distributed across the scalp (FIG. 3). The trial number was again 100. The weight distributions of S and R components are presented in FIG. 3. Pink noise was independently generated and added to each single trial and channel with the same strength (σ₂). Consider the following four different cases of simulation data for Model 2:
(1) Case 1: S and R have different waveforms and the same spatial weight distribution (FIG. 3A), σ₁=80, σ₂=0.1.
(2) Case 2: S and R have the same waveform but different spatial weight distribution (FIG. 3B), σ₁=80, σ₂=0.1.
(3) Case 3: S and R have different waveforms and different spatial weight distribution (FIG. 3C), σ₁=80, σ₂=0.1.
(4) Case 4: the same as case 3 except that R has no latency jitter (FIG. 3D), i.e., σ₁=0, σ₂=0.1.

Experiment

Two published experimental datasets were used for method comparisons. Data Set 1 was taken from Herzmann, G., & Sommer, W. (2010). Effects of previous experience and associated knowledge on retrieval processes of faces: An ERP investigation of newly learned faces. Brain research, 1356: 54-72 (the content of which is incorporated herein by reference in its entirety), where specific details can be found. In short, data were collected from 21 participants who performed a speeded familiarity decision tasks on a set of familiar and unfamiliar faces. Each familiar and unfamiliar face was preceded either by itself (primed) or by a different face (unprimed). EEG was recorded from 65 electrode channels. Participants indicated target familiarity by key presses with their index fingers. The assignment of familiar and unfamiliar stimuli to the left or right hand was counterbalanced.

Data Set 2 was taken from Bayer, M., Sommer, W., & Schacht, A. (2012). P1 and beyond: Functional separation of multiple emotion effects in word recognition. Psychophysiology, 49:959-969 (the content of which is incorporated herein by reference in its entirety), where details can be found. In short, data were obtained from 23 participants who performed a lexical decision task, that is, they decided whether a given letter string represented a real German word or not (i.e., was a pseudoword). EEG was recorded from 61 electrode channels. All responses were made with the right hand.

Results

In this section, firstly presented are the results of the comparison of RIDE with the existing temporal and spatial decomposition methods using simulation data according to Model 2. Then it is shown the results of applying RIDE to real data to obtain the component clusters, to reconstruct the ERP from latency-corrected components and to explicitly validate Model 2 by examining the reliability and variability of the components in single trials. Lastly, RIDE and ICA in real data are briefly compared.

Comparison of RIDE with Temporal and Spatial Decomposition Methods in Simulation Model

Divergence and Distortion in Temporal Decomposition Methods

Since all previous temporal decomposition methods are mathematically equivalent, Fourier decomposition for the comparison with RIDE is applied (FIG. 2). The latency-variable component R can be severely smeared in the conventional stimulus-locked average ERP due to strong latency variability (FIG. 2 B). It is shown typical separation results for four different extents of latency variability of R components in the four rows of FIG. 2 II. The single trial simulation data sorted by the latency of R is shown in the left panel of FIG. 2 II for different σ₁ (100, 50, 20, and 3). The separated S and R components from Fourier decomposition and RIDE are shown in the middle and right panels of FIG. 2 II, respectively.

Consistent with our theoretical analysis in Eq. (7), especially in the low frequency range the noise for the Fourier decomposition, is amplified and seriously distorts the waveforms of S and R as the latency distribution of R narrows (FIG. 2 D, G, J). Fourier decomposition showed divergence when σ₁ approached small values, creating two opposite and complementary components with huge amplitudes (FIG. 2 M). In sharp contrast, RIDE with median waveform faithfully recovered the S and R components without low-frequency distortion (FIG. 2 E, H, K). More importantly, RIDE did not yield divergent results when the latency jitter shrank to very small values. With decreasing σ₁ RIDE appeared to allocate increasing portions of the composite ERP to the S component, and in the limiting case when the latency jitter approached zero, RIDE yielded only one ERP component cluster. This is consistent with the situation in the simulation data: at the limit of infinitesimal latency variability, S and R are locked to each other (and to the stimulus) in time and are correctly treated as one single stimulus-locked component cluster (FIG. 2 N). This is in sharp contrast to Fourier decomposition, which generates totally incorrect results for the case of small latency variance (Figure M). FIG. 2Q showed that RIDE achieved convergence quickly within about just ten steps.

Next, the performance of RIDE is compared more systematically with the Fourier decomposition with respect to latency variability σ₁ and noise strength σ₂. Performance was evaluated by the decomposition error (FIG. 2P, R), defined as the summed square of the difference between the separated components and the real components put into the model. As expected, Fourier decomposition showed large errors in a broad region and displayed divergence for small σ₁ and large σ₂ (sharp bright region in FIG. 2 P). The errors have larger power in the low frequency domain (FIG. 2 O), consistent with Eq. (7). In contrast, RIDE did not show divergence in the corresponding region and yielded much smaller error in the whole parameter range (FIG. 2 R) (note that the grey scale is already ⅕ of that in FIG. 2 P).

Further description of FIG. 2 is: Upper Panel I: A: Illustration of the simulation data for single trial ERPs. B: The conventional stimulus-locked average ERP from N=100 simulated single trials, with noise strength σ₂=0. 5. Middle Panel II: Typical examples of separation results for different latency variability σ₁ of R component. C, F, I, L: Grey scaled plots of all single trials of the simulation data (sorted by latency of R) for different σ₁. D, G, J, M: The separated S and R components from Fourier decomposition. E, H, K, N: The separated S and R components from RIDE. Lower Panel III: performance comparison: O: The power of the difference between the separated S and given S with respect to σ₁ for different frequencies k by the Fourier decomposition. P: The standard deviation of the error of the separated results (averaged from both S and R) across different values of σ₁ and σ₂ for Fourier decomposition. Q: The convergence of iteration in RIDE. The plot shows the peak value of R component during the iteration (from E). R: the same as P, but for RIDE. Note the different scales in the grey scale bars for P and R.

The amplification of low frequency noise can be witnessed in several previous publications about ERP decomposition methods in both simulations as well as real data such as Hansen, J. C. (1983). Separation of overlapping waveforms having known temporal distributions. Journal of neuroscience methods 9:127-139 (the content of which is incorporated herein by reference in its entirety) and Takeda, Y., Yamanaka, K., & Yamamoto, Y. (2008). Temporal decomposition of EEG during a simple reaction time task into stimulus-and response-locked components. NeuroImage 39:742-754 (the content of which is incorporated herein by reference in its entirety). Although some simple techniques have been used to alleviate this problem such as low-pass filtering and linear de-trending, they could not completely remove the distortion. RIDE solves the problem by simply using the median wave instead of the average (L1 instead of L2 norm minimization). Rather than giving rise to diverging complementary components as in the previous temporal decomposition methods, RIDE tends to group ERP components with weak latency variability to a single component cluster, which is the proper way to view these components. To summarize, RIDE can firmly establish the ERP Model 2 and is compatible to the conventional Model 1 if all components are indeed locked in time.

Comparison of RIDE with ICA

RIDE and ICA decompose ERPs from different perspectives and are based on different theoretical frameworks. A component cluster defined in RIDE is the ensemble of ERP components that are locked to the same time event. RIDE rests on the main assumption that the timing of each component cluster in each single trial is known or can be reliably estimated and there is significant latency jitter between the component clusters. The components defined in ICA are the independent brain sources that generate and project signals to the scalp; ICA rests on the main assumption that different sources have different spatial weights and are statistically independent of each other. Here, the applicability of RIDE is concisely compared with ICA in the simulated data according to Model 2 with several representative situations. These examples include some extreme situations to clearly reveal the different utility of RIDE and ICA. The results are shown in FIG. 3 A-D for 4 different cases of simulation data sets 1-4, respectively. The left column shows the waveforms and spatial weights of the components in the simulation model, the middle column presents the decomposition results from RIDE, and the right column contains the topographies of the two strongest sources separated by ICA and their activations; 17 further noise-like sources obtained by ICA are not shown.

In FIG. 3, there is presented the comparison of RIDE and ICA in the simulation data. Further description of FIG. 3 is: A, B, C, D: Simulation data in cases 1, 2, 3, and 4, respectively. Left column: time courses of S and R and their topographies at the peak (time courses are from the electrode with the strongest activity). Middle column: time courses of S and R separated by RIDE and their topographies at the peak. Right column: time courses of S and R (first two ICs) separated by ICA and the corresponding weight distribution. E: the locations of the virtual electrodes across the scalp. F: residual error per electrode (calculated as summed square of errors averaged over S and R and electrodes) of the separated components by RIDE and ICA with respect to σ₁ for the simulation data case 2. The black line is error induced solely by the background noise.

As can be seen in FIGS. 3A and 3B, ICA does not separate components that have the same spatial distribution but different event-locking; it also does not work when sources have distinct spatial weights and significant latency jitter but highly correlated waveforms. In these two cases, the requirements of spatial and temporal independence of the signals for ICA are not satisfied. RIDE only makes use of the latency variability and is able to separate components irrespective of their scalp distributions and waveforms. Though the extreme case of identical waveform of S and R in FIG. 3B is used, the conclusions can be extended to non-identical but correlated waveforms (data not shown). In real ERP data, the situation is actually tougher for ICA as, for example, the sub-components of the P3 family could have both similar topographies and correlated waveforms. The difficulty of ICA with highly correlated sources was also mentioned by Makeig et al. in Makeig, S., Jung, T. P., Bell, A. J., Ghahremani, D., & Sejnowski, T. J. (1997). Blind separation of auditory event-related brain responses into independent components. Proceedings of the National Academy of Sciences, 94:10979-10984 (the content of which is incorporated herein by reference in its entirety). Interestingly, strong latency variability of component cluster R can effectively induce the independence between S and R for the case in FIG. 3B, enhancing the performance of ICA. FIG. 3F shows the dependence of the summed square error of the separated waveforms of S and R as a function of latency variability σ₁. When σ₁ is close to zero, both ICA and RIDE extract one noise-free component instead of two components, giving rise to the same error. RIDE can separate the two components reliably when the variability becomes large enough (˜50), but ICA can only disentangle the two sources at much larger latency variability (˜100). However, the R component separated by ICA always remains smeared, hence the error of ICA does not drop to the noise level as for RIDE.

FIG. 3C shows that both ICA and RIDE can be used to separate components with distinct spatial weights, highly uncorrelated waveforms and significant latency variability, although they are based on different theories. In this case, the properties of S and R satisfy both the conditions of RIDE and ICA, and both methods can separate them although RIDE utilizes the property of latency variability while ICA utilizes the property of independence. However, it is necessary to point out that ICA does not intrinsically utilize the latency variability information of R, hence the resulting waveform of R is the average over single trials with respect to the stimulus onset and is actually again a smeared shape of R convoluted by the latency distributions (FIG. 3C, right). In contrast, the waveform of R separated by RIDE using latency information neatly recovers the true un-smeared waveform and topographies.

FIG. 3D shows the strength of ICA to separate spatially distinct sources, which are time-locked from trial to trial while RIDE would allocate them to the same component cluster. As elaborated in the previous section, RIDE considers spatially distinct components locked to the same time event (e.g., stimulus onset) as the same component cluster. As a result, the summation of S and R components will be allocated to S, leading to zero contribution to R (FIG. 3D, middle). Conversely, the conditions for ICA are satisfied by distinct spatial distributions and time courses, hence the components can be separated although they are temporally locked to each other from trial to trial (FIG. 3D, right).

Separation of Experimental Data by RIDE and the Psychological Implications

RIDE is applied to two different experimental datasets to show that the new paradigm of ERP model with latency variable components can be used to restore ERP components that are smeared and blurred or even hidden in the standard averaged ERP due to latency variability. With latency-corrected components, the ERP can be reconstructed and amplitudes in late time windows can be significantly enhanced relative to the standard average ERP. Furthermore, the model is directly validated by examining the reliable presence of the components in the single trials on the one hand, but highly variable in the amplitude and latency on the other hand. The outcomes of RIDE, that is, reconstructed ERP, separated component clusters, and single trial variability information can be used to study the brain-behavior relationships, with much more information than provided by the conventional average ERP. A comparison of RIDE with ICA on real data is made to further demonstrate that ICA is not suitable for Model 2 in real data.

Component Decomposition and Restoration

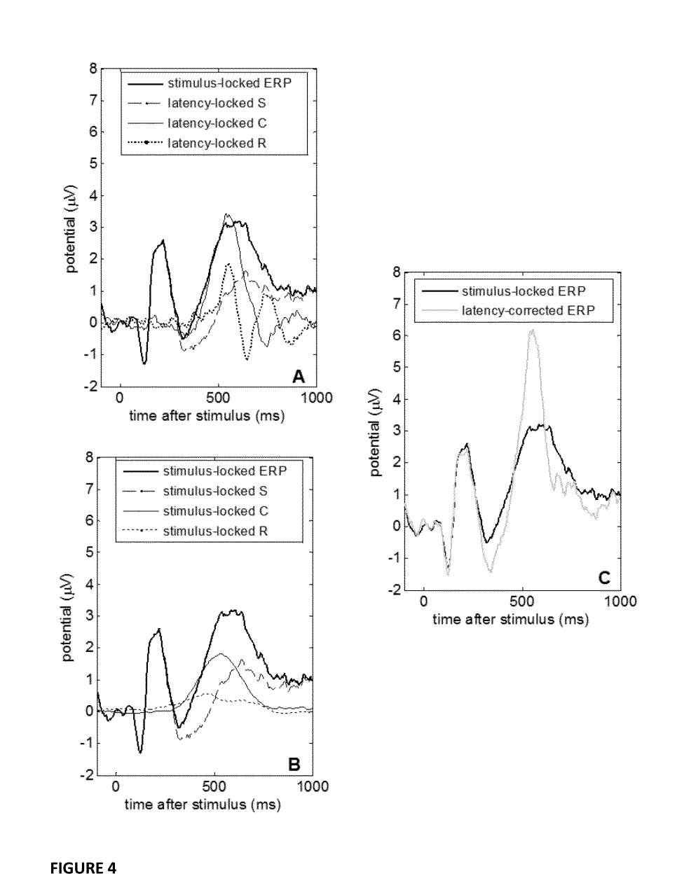

FIG. 4 shows the result of applying RIDE to the ERP dataset from the lexical decision task. The wave shapes are grand averages over 23 participants. In FIG. 4 the data is from one condition (neutral valence, high arousal) of the lexical decision task. The grand mean RT for this condition was 607 ms. Results are shown for channel Cz. A: Grand average time course of ERP and RIDE component clusters synchronized to the most probable latency. B: Grand average time course of ERP and RIDE component clusters, stimulus-synchronized (ERP and S do not differ from plot A). C: Grand average stimulus-locked ERP and the reconstructed (latency-corrected) ERP (summation of RIDE components from plot A). FIG. 4A shows the original stimulus-locked ERP for electrode Cz and the RIDE components separated from the ERP. The time courses of RIDE components were averaged after firstly synchronizing to their most probable latency across single trials and then compensating the subject-to-subject variability (C peak was synchronized to the mean value of the most probable peak latency and R was synchronized to the mean RT of all participants). The RIDE results reveal a much richer internal structure of ERP components, which are hidden in the original average ERP. To more clearly visualize this startling smearing effect by latency variability, the stimulus-locked RIDE components S, C_sl and R_sl are plotted in FIG. 4 B, in which the RIDE components S, C, and R were distributed back to their single trial latencies with respect to stimulus onset and averaged over trials (S in FIGS. 4A and 4B is the same since S is locked to stimulus onset). Note that in FIG. 4B, the conventional average ERP is related to the stimulus-locked RIDE components as ERP=S+C_sl+R_sl. In sharp contrast to the latency-locked RIDE components in FIG. 4A, the stimulus-locked RIDE components C and R are now seriously blurred and flattened, especially for the R component. Once the components and their most probable latencies (FIG. 4A) are obtained, the ERP from the smearing effects of latency variability according to Eq. (4) can be restored. Such a reconstructed ERP obtained by summing the latency-corrected ERP components in FIG. 4A is shown in FIG. 4C, in comparison with the conventional stimulus-locked averaged ERP. FIG. 4C visualizes how strongly the amplitude and pattern of the average ERP is blurred by latency variability. In the early time window they do not differ from each other since latency variable components C and R only start to emerge in late sub-processes. Astonishingly, in the late time window, the amplitude of the latency-corrected ERP can double that of conventional stimulus-locked averaged ERP. This example demonstrates the need to deal with latency variability in ERP analysis, since it could lead to serious misinterpretations of amplitude effects; for example, condition effects in ERP amplitudes might be induced by different amounts of latency variability. After reconstruction by RIDE the ERP can be used in the same way as a conventional ERP.

Functional Relevance of the Separated Components

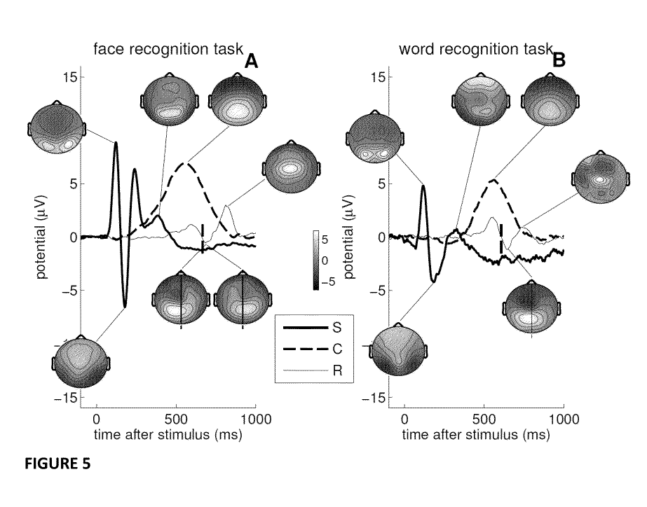

The psychological significance of the RIDE-separated components and their consistency can be examined by considering the waveforms and topographies across different datasets. When brain processes in different cognition tasks, with certain task-specific component differing across tasks, some sub-processes such as perception, stimulus evaluation, and motor response should still be reliably present and separable to different component clusters. In FIG. 5 several representative topographies are shown for the components S, C and R respectively, for a face recognition (A) and a lexical decision task (B). The electrodes PO8, Pz and Cz were selected to show the curves of S, C and R, respectively. Maps show scalp distribution for selected peaks and troughs. Here in face recognition task, there were responses by left-hand and right hand, and the topographies of R for right-handed and left-handed responses show asymmetrical patterns and in word recognition task there were only responses by right-hand and the pattern is consistent with the right-hand response in face recognition. The vertical dashed lines indicate the grand mean RT. As shown in FIG. 5, the components separated by RIDE reliably reflect the specificity and consistency of waveforms of neurocognitive activity across subjects, conditions and even experiment paradigms. FIGS. 5A and 5B show, in order, the grand average RIDE components from the face recognition and lexical decision task. The three curves are for S, C and R component clusters at their most prominent channels, respectively. The topography of the RIDE component clusters at some representative time points is selectively shown.

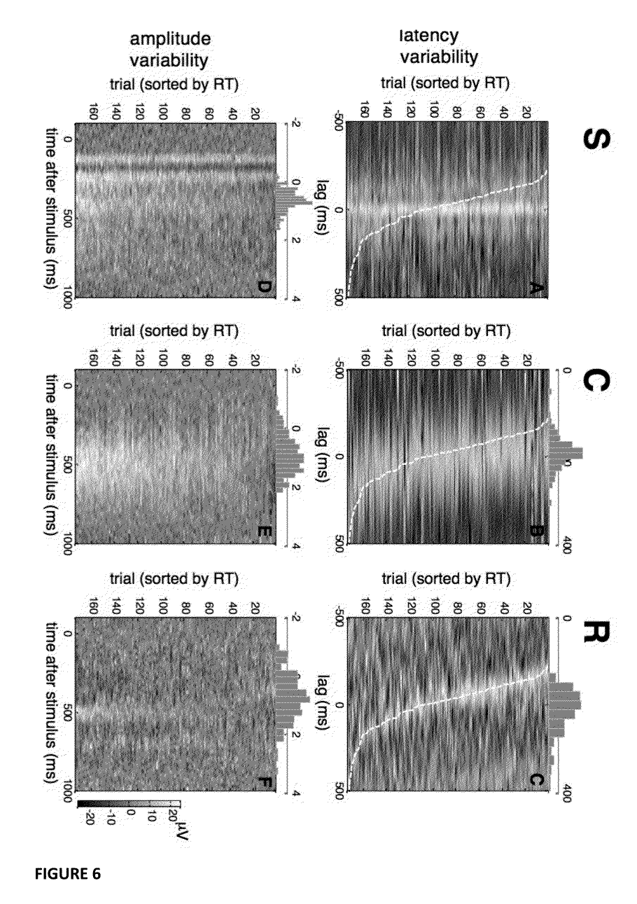

Generally, the patterns of the time courses and topographies (at some representative peaks and troughs) of RIDE components show high consistency across the two different tasks. The results are selectively shown on some electrodes and time points but this consistency is generally seen across the scalp and time (data not shown). Specifically, several interesting points may be noted in FIG. 5:
1. The S component cluster capturing most of the stimulus-locked components, such as the P1 and N1/N170 with topography positivity at O1 and O2.
2. S also captured a small portion of the P3 complex elicited by faces, as can be seen in the topography of S at the third peak. The main portion of the P3 complex was captured by the C component cluster and P3 latency correlated with RT (FIG. 6B).
3. The R cluster captures components with relatively weak amplitudes but high frequency of oscillation. Notably, this motor related R component cluster that is mostly blurred in original ERP shows consistent waveforms and hand-related asymmetries across tasks. For the face task with left- and right hand responses the topographies of the component immediately following the response is roughly mirror symmetric for the hands (FIG. 5A, bottom, second and third topographies); the right-hand responses in the lexical decision task (FIG. 5A, bottom, second topography) show a similar topography as the right-hand responses for the face task. Importantly, the latency of R showed response-locking (see FIG. 6 C also).

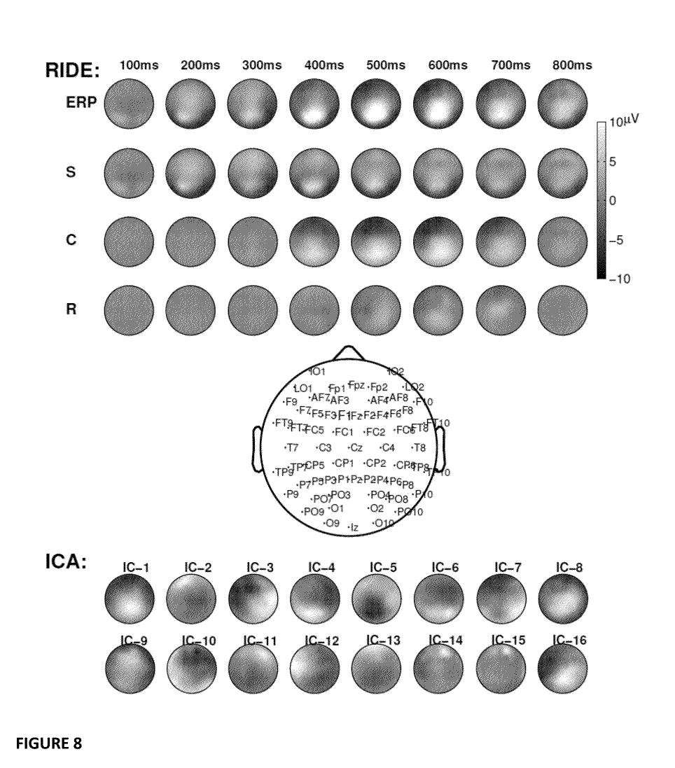

These results indicate the functional relevance of the separated components to cognitive sub-processes and events. The evolution of the topography of the RIDE components can also be seen in FIG. 8 when compared to the ICs from ICA.

On the one hand, the consistency of waveforms and topographies across different tasks confirms that the new Model 2 is applicable to separate ERPs into three major component clusters. On the other hand, there are also some task- and material-specific findings that can be most likely attributed to the different underlying neural mechanisms for different tasks. The P1 is seen to reflect visual processes in extrastriate areas and the topographies are very similar in both data sets; the amplitude difference is naturally explained by the larger patterns of faces than words. The N1/N170 is usually larger in faces than in other stimuli and represents the structural analysis of faces and words. The topographies show the typical right- and left-hemispheric asymmetries of the N170 for faces and words, respectively.

The topographies of P3 in S and in C to face stimuli show somewhat distinct patterns, with more localized and more broadly distributed positivities in S and C, respectively. These results indicate that the S and C portions of P3 reflect different neurocognitive processes; one might speculate that the stimulus-locked part is triggered by the intrinsic salience of the face stimuli, whereas the P3 in the C component for both faces and words reflects more time-variable aspects of task-relevance. The hand-related asymmetries for the R component reflect the expected motor activation during preparation and execution of manual responses.

Together, the present application of the extended RIDE to real data sets show that separated components can be used to investigate more deeply both task-general and task-specific features. Furthermore, the neural sources of ERPs, especially late components, might be more precisely localizable after applying RIDE.

As demonstrated in the simulation section, L2-norm based methods amplify the slow-frequency noise whereas the L1 norm based algorithm of RIDE avoids such distortion. Therefore L1 norm based method should also generate more consistent results across subjects. To show this in real data, the consistency of RIDE separation across subjects for both L1 and L2 algorithms is quantified by calculating the standard deviations of the time course across participants (after averaging over S, C and R and all electrodes within each participant). The results in Table 1 show that the L1 norm based RIDE method show higher consistency (i.e., smaller standard deviations) across participants than the L2 norm based algorithm, confirming that L1 norm minimization is preferable.

TABLE 1
Comparison of the consistency of L1 and L2 norm algorithms.

| Cross-subject consistency | |
|---|---|
| Word | Face |
| recognition | recognition |
| task | task |

| | L1 | L2 | L1 | L2 |
|---|---|---|---|---|
| Standard deviation | 1.30 | 1.47 | 1.36 | 1.57 |

Trial-to-Trial Variability and Reliability of the ERP Components

It is assumed that a sequence of cognitive sub-processes are carried out as the brain accomplishes a task and these sub-processes should be present in repeated trials of the same type. Therefore, the ERP components associated with such sub-processes are expected to be reliably visible in each single trial. Nevertheless, there is variability in amplitude and latency from trial to trial. RIDE allows to explicitly incorporating this new concept by integrating reliability of processing with variability in strength and latency of the brain response to stimulus. The analysis below on both the variability and the reliability of the components across single trials provides direct and strong validation of the Model 2 as a new paradigm of ERPs.

RIDE exploits trial-to-trial variability in order to obtain the ERP components associated with different cognitive functions and restore the ERPs from smearing. RIDE can thus provide rich information about the variability of brain responses across single trial ERPs. Each component cluster may have its own variability in latency and amplitude. By matching with the component template, both the latency and amplitude of each component cluster in each trial can be estimated (FIG. 6).

For estimating latencies in the present demonstration, cross-correlation between the templates of the RIDE component clusters and single trial ERPs after removal of the other component clusters is used (here the template was calculated after excluding the trial in question to avoid any self-contribution effect in the cross-correlation). The cross-correlation curves were averaged across all electrodes. FIG. 6A-C show the cross-correlation curves for S, C, and R, respectively. The peak for each single trial distribution corresponds to the relative shift of the RIDE component cluster in the trial with respect to the template. Consistent with the assumption of RIDE, the latencies of S and R are tightly locked to stimulus (FIG. 6A) and response (FIG. 6C), respectively, whereas the latencies of C vary strongly and are also correlated with the response (FIG. 6B, the correlations differ between participants). To show the amplitude variability across single trials, the amplitude of each RIDE component of each single trial is estimated by calculating the covariance between the template and single trial (after removal of the other component clusters). After estimating the amplitude, representative electrodes for different components are selected to show the amplitude variability by sorting the original single trial ERP according to the estimated amplitudes (FIG. 6D-F). It can be seen that there are strong fluctuations of amplitudes for each RIDE component cluster across single trials. The trial-to-trial analysis shows that the components are present in single trials, and the corresponding component templates obtained by RIDE correspond to their most probable amplitude and latency in the single trials. Thus, the reconstructed ERP from latency-corrected components restored by RIDE from the smeared average ERP represents the most probable waveform that can be observed in single trial ERPs. The Model 2 in Eq. (1) thus does not need to explicitly consider amplitude variability.

FIG. 6 shows the latency and amplitude variability of RIDE component clusters across single trials for one participant from the primed-unfamiliar condition of the face recognition experiment. A-C: The cross-correlation between each RIDE component cluster (S, C, R) and the single trials after removal of the other component clusters (maximal value normalized across trials for clear presentation). The trials were sorted by RT (white dashed line). The histograms above B and C show the distributions of latency of C component and RT. D-F: The single trial ERP data of each RIDE component cluster sorted according to the estimated amplitude. The trials were synchronized to the most probable latency of the corresponding components. S, C, and R are from channel PO8, Pz, and Cz, respectively. The histograms above D-F show the distributions of the amplitudes of S, C, and R across single trials estimated by the covariance between the template and single trials (without the other two components), normalized by the variance of the template. Thus covariance value 1.0 corresponds to the amplitude of the component template.

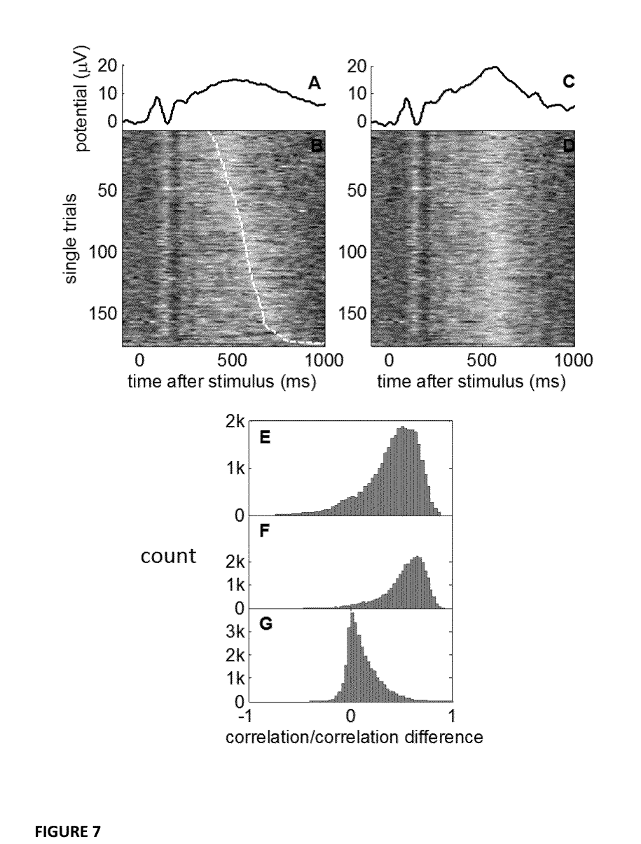

Despite the strong variability in both latency and amplitude, the components are reliably present in most of the single trials. Here, more quantitative evidence of this reliability is presented. FIG. 7B shows the single trial ERP from one electrode of the same subject as in FIG. 6. The latency variability of the response-related component is clearly visible. The pair-wise correlations between the single trials are mainly positive, but there is also a significant portion of negative values (FIG. 7E). After RIDE decomposition and re-locating each component to their most probable latency (FIG. 7B), the distribution of the pair-wise correlation of such latency corrected single-trails shift to the right as a whole and becomes more narrowly distributed at larger values (FIG. 7F), with most of the pairs improved in correlation, that is, reliability (FIG. 7G). The comparison between the conventional stimulus-locked averaged ERP in FIG. 7A and the reconstructed ERP from latency-corrected components in FIG. 7B shows again that, similar to FIG. 4C for the word recognition task, the latency variability can strongly diminish the amplitude of average ERP in late time window of the face recognition task.

In particular, FIG. 7 shows the trial-to-trial reliability (the selected channel was PO4). The data is the same as FIG. 6 for the face recognition task. A: The stimulus-locked average ERP. B: The single trial ERP sorted by RT (amplitude normalized for better visualization of the response locked components). C: The reconstructed ERP from latency-corrected components. D: The latency-corrected single trials corresponding to B. E and F: Distributions of all pair-wise correlations between single trials from B and of the latency-corrected single trials from D, respectively. G: Distribution of the differences of pair-wise correlations from D plot and B plot.

The above results show that the components are reliably present in most single trials but can be highly variable in latency. This new picture of brain processing supports Model 2 and shall provide a new paradigm to understand brain-behavior relationship.

Compare RIDE with ICA in Real Data

RIDE separates ERPs into component clusters associated with different events and ICA separates ERP into independent components representing sources of brain activity. RIDE is applied to the data of one participant from the face recognition task. The topography evolution of the standard average ERP and the RIDE-separated components is shown in FIG. 8, top panel. It can be seen that 1) the early visual components (before ˜200 ms) are completely located in the S component cluster; 2) LPC (Late Positive Components, 300-800 ms) are allocated to S and C, and C component captures the bigger and more parietal portion (P3b) and S captures the centrally distributed portion; 3) The R component cluster captures most of the motor-related processes because it displays the activities above the motor region with significant hand-related asymmetry. In particular, FIG. 8 shows the comparison between RIDE and ICA in real data from a single right-handed subject of the face recognition task, wherein Top: the temporal evolution of the topographies of ERPs and RIDE component clusters. Here the components are located at the corresponding most probable latencies as done when reconstructing the ERPs, wherein Middle: Locations of the electrodes, and Bottom: The first 16 most significant ICs separated by ICA.

ICA separates ERPs into as many components as there are electrodes. Note that the present data subjected to ICA decomposition was pre-processed after standard artifact rejection. The components show different topographies (FIG. 8 bottom), which may also differ across participants, rendering the selection of the appropriate components and the psychological interpretation decidedly non-trivial. On the contrary, all the above results showed that the components obtained by RIDE are consistently present with interpretable psychological relevance and more likely to capture different sub-processes such as stimulus perception, decision-making and response execution.

Summary

Summary and Potential Applications of the New Paradigm of ERP Analysis with RIDE

It has been recognized long time ago that the timing of components within an ERP is not fixed but varies and the variability may accumulate. However, the issue has been mostly ignored or addressed only imperfectly, rendering the conventional stimulus-locked average ERP to overwhelmingly dominate neurocognitive ERP research. Traditionally, there have been several attempts to deal with the problem. The first was Woody filtering in 1967, Characterization of an adaptive filter for the analysis of variable latency neuroelectric signals. Medical and biological engineering, 5: 539-554 (the content of which is incorporated herein by reference in its entirety), which focused on the central component (e.g., P300), but ignored the other components. A further attempt is response-locked averaging, which focuses on the R-component but blurs the C- and S-components. Traditional decomposition methods such as Hansen, J. C. (1983). Separation of overlapping waveforms having known temporal distributions. Journal of neuroscience methods 9:127-139 (the content of which is incorporated herein by reference in its entirety), and Zhang, J. (1998). Decomposing stimulus and response component waveforms in ERP. Journal of neuroscience methods 80:49-6 (the content of which is incorporated herein by reference in its entirety) try to separate the stimulus-locked and response-locked components, but are unable to deal with central components without explicit time markers. ICA can potentially separate independent sources but is not supposed to be suitable to deal with the temporal variability problems described in Model 2.

The present invention systematically presented the extended RIDE as a new method that can integrate the advantages of the previous methods and overcome several major limitations, establishing a model of latency-variable components in ERPs as a new paradigm for ERP analysis. The new paradigm simultaneously captures two essential features of neurocognitive processing, being composed of several subprocesses, which are carried out reliably but with more or less trial-to-trial variability in latency and amplitudes of the corresponding ERP components. The linear superposition Model 2 in Eq. (1) allows for the decomposition and reconstruction of the ERP components and the analysis of single trial variability in both amplitude and latency. It is provided direct and explicit validations of the new model in real data sets by the reliable presence of the subprocesses and the associated components and their variability in latency and amplitude across single trials (FIGS. 6 and 7) and demonstrated the functional relevance of the separated components (FIG. 5). Similar validations of the model and the functional relevance of the RIDE components were also provided in previous applications of RIDE.

Two innovative aspects of the extended RIDE allow establishing the new paradigm of ERP analysis. 1) RIDE involves a latency estimation scheme for components without explicit latency information. This also allows applications to data without responses (nogo, counting, reading). 2) RIDE uses iteration to obtain median waveforms to realize L1 norm minimization to prevent slow wave amplification and distortion typical for conventional temporal decomposition methods.

### FURTHER EMBODIMENTS OF THE PRESENT INVENTION

The new paradigm of ERP analysis and its implementation with RIDE could lead to several new directions of cognitive studies with ERPs:

i) Extract components associated with specific cognitive subprocesses. Both in time course and topography, RIDE can be applied to extract components either mixed with each other or blurred by latency variability (FIG. 4), or to separate exo- and endogenous components and specific cognition-related component (e.g, N200). The separated ERP components allow us to study experimental effects on different subprocesses, which would potentially provide a deeper understanding of the neural mechanisms of brain processing and cognition. The purified component topography from mixing and smearing should be very helpful for better and more precise localization of the cortical sources, which might significantly improve the dynamical causal modeling of the processing networks.

ii) Reconstruct ERP from latency-corrected components. By compensating the latency variability and hence restoring the ERP effects by the summation of each RIDE-decomposed component at their most probable latency across single trials the reconstructed ERP represents the most probable waveform in single trials. The reduced amplitude of the conventional ERP components in late time windows due to trial-to-trial variability can be very significant and may now be corrected. In certain situations, the diminished amplitude in averaged ERPs may lead to statistically insignificant conditional effects and impede understanding of brain-behavior relationships. After reconstruction from latency variability clearer conclusions may become possible. On the other hand, the presence of significant amplitude effects in average ERPs may be due to differences in latency variability between conditions; for example, amplitude differences as a function of task difficulty might be due to larger latency in a more difficult condition leading to amplitude attenuation in the average ERP, while the amplitude of the component in single trials may be unaffected. Similar issues may arise when comparing different population samples, for example, of different age or healthy status. The assessment of RIDE-reconstructed ERPs would contribute to clarify these issues.

iii) Study trial-to-trial variability. In addition to extracting component clusters, RIDE also allows to obtain component amplitudes and latencies in single trials. For example, the latency of C component obtained by RIDE shows significant trial-to-trial variability, which is correlated with RTs to different extents across individuals and conditions. This information of trial-to-trial variability could serve as valuable indicators reflecting brain's dynamical working mechanism in connections with internal background activities and external experimental factors.

iv) Study individual differences, aging and diseases. The trial-to-trial variability within a subject may be closely related to individual differences on the behavioral performance and neural level. Aging and also diseases may not only cause amplitude decrements in ERPs but also increase variability in ERP latencies and reaction times. With the option to obtain both the components and variability information, RIDE could be very valuable to analyze ERPs in studying individual difference, aging and disease.

### III. Conclusions

Though it has recognized that latency variability of the ERP components in single trials could cause serious problems of mixing and smearing the average ERP waveform and topography, previous attempts have not succeeded in establishing the latency-variable model as a well-accepted and widely applied paradigm for ERP analysis. Here, the major limitations in previous methods are identified and a new method (RIDE) is presented, which combines the advantages but overcome the limitations of previous methods, firmly establishing the new paradigm of ERP analysis. With the new framework assuming temporally overlapping components with variable latencies and associated to different events with or without explicit time markers, RIDE allows to extract the ERP components for particular sub-processes. RIDE allows to reconstruct ERP waveform as can be most probably observed in single trials and to obtain the distributions of latency and amplitude of each components among single trials. By applying RIDE, EEG data can now be explored in much broader scope to study the brain-behavior relations than the conventional average ERP allows, where especially cognition-related components may be strongly smeared and where the valuable dynamical information in single trials is lost.

### APPENDIX

Analysis of the distortion and divergence in previous temporal decomposition methods

The final form of the least square solution to (1) is:

C(k)=(L^{T}(k)L(k))⁻¹L^{T}(k)EEG(k) (A1)

where EEG(k) is the signal matrix, C is the component matrix, L is the coefficient matrix containing the information of latencies of each components, k is the frequency. Every term in (A1) is the representation after Fourier Transform.

In spite of its closed-form, it is shown that solution (A1) has noise-induced divergence property when latency jitter between each two components shrinks. For the case of two components locked to different time-markers (e.g. stimulus-locked component S and response-locked component R), the EEG trials are embedded with noise and the trial number is adequately high (to fulfill the approximation in the analytical derivation below), the solution of S and R from (A1) is:

$$\begin{matrix}{S}^{′ ( k )} = \frac{〈 {EEG}_{{S}_{i}} ( k ) 〉 - 〈 {EEG}_{{R}_{i}} ( k ) 〉 \cdot 〈 {e}^{ck {τ}_{i}} 〉}{1 - {\vert 〈 {e}^{ck {τ}_{i}} 〉 \vert}^{2}} , & ( A2 ) \\ {R}^{′ ( k )} = \frac{〈 {EEG}_{{R}_{i}} ( k ) 〉 - 〈 {EEG}_{{S}_{i}} ( k ) 〉 \cdot 〈 {e}^{- ck {τ}_{i}} 〉}{1 - {\vert 〈 {e}^{ck {τ}_{i}} 〉 \vert}^{2}} . & ( A 3 )\end{matrix}$$

where c=−2πj/M, M is the number of sampling points for a single trial, j is the complex unit, EEG_Sᵢ is the single trials locked to stimulus onset and EEG_Rᵢ is single trials locked to response times. Assumed are a precise S component and R component summed by random noise with fixed magnitude A and uniformly distributed phases θ on [0, 2π], thus:

$$\begin{matrix}{EEG\_S}_{i} ( k ) = S ( k ) + R ( k ) \cdot {e}^{ck {τ}_{i}} + A \cdot {e}^{ckt + j {θ}_{i , k}} , & ( A4 ) \\ {EEG\_R}_{i} ( k ) = R ( k ) + S ( k ) \cdot {e}^{- ck {τ}_{i}} + A \cdot {e}^{ck ( t + {τ}_{i} ) + j {θ}_{i , k}} . & ( A 5 )\end{matrix}$$

Substitute (A4) and (A5) to (A2):

$$\begin{matrix}{S}^{′} ( k ) = S ( k ) + A \cdot {e}^{ckt} \cdot \frac{〈 {e}^{j {θ}_{i , k}} 〉 - 〈 {e}^{ck {τ}_{i} + j {θ}_{i , k}} 〉 \cdot 〈 {e}^{ck {τ}_{i}} 〉}{1 - {\vert 〈 {e}^{ck {τ}_{i}} 〉 \vert}^{2}} & ( A6 ) \\ = S ( k ) + ɛ ( k ) , & ( A 7 )\end{matrix}$$

where the second term is erroneously estimated part, namely the error term ε. Without loss of generality, set A=1, c=j, therefore the magnitude of the error term becomes:

$$\vert ɛ ( k ) \vert = \vert \frac{〈 {e}^{j {θ}_{i , k}} 〉 - 〈 {e}^{j ( k {τ}_{i} + {θ}_{i , k} )} 〉 \cdot 〈 {e}^{j k {τ}_{i}} 〉}{1 - {\vert 〈 {e}^{jk {τ}_{i}} 〉 \vert}^{2}} \vert . \qquad ( A8 )$$

Use the properties of the production of two random variables X and Y: <XY>=<X><Y>+cov(X,Y)=<X><Y>, when X and Y are uncorrelated:

$$\vert ɛ ( k ) \vert = \vert 〈 {e}^{j {θ}_{i , k}} 〉 \vert \cdot \vert \frac{1 - {〈 {e}^{jk {τ}_{i}} 〉}^{2}}{1 - {\vert 〈 {e}^{jk {τ}_{i}} 〉 \vert}^{2}} \vert . \qquad ( A9 )$$

To prove that the magnitude of ε(k) diverges as the variance of τ shrinks to zero, we only need to show that the second term in the right hand side of (A9) diverges as the variance of τ shrinks to zero. It can be shown that the imaginary part of the second term (inside part) of (A9) diverges:

$$imag ( \frac{1 - {〈 {e}^{jk {τ}_{i}} 〉}^{2}}{1 - {\vert 〈 {e}^{jk {τ}_{i}} 〉 \vert}^{2}} ) = \frac{2 〈 cos k {τ}_{i} 〉 〈 sin k {τ}_{i} 〉}{1 - {〈 cos k {τ}_{i} 〉}^{2} - {〈 sin k {τ}_{i} 〉}^{2}} \qquad ( A10 )$$

Assume the set of τᵢ is Gaussian distributed. Let all the value of τᵢ be fixed, shrink the set of τᵢ by attaching each τᵢ with a coefficient σ, replace the trigonometric functions by Taylor series and retain the terms with first order (analyzing the property of the function when a approaches zero). The result of (A10) becomes:

$$\frac{Const}{σ {k}^{2}} \qquad ( A11 )$$

where σ is the shrinking parameter (represents the variance of τ). The term (A11) will diverge when σ approaches infinitesimal, that is, the noise-induced error term ε shows divergence property with narrowing of the distribution of τ, namely the latency jitter.

### II. Flow Chart of Ride and Additional Technical Issues

Flow chart of RIDE is shown in FIG. 9.

### INDUSTRIAL APPLICABILITY

The present invention relates to a method for separating and analyzing overlapping data components with variable delays in single trials. In particular, the present invention relates to a method for separating and analyzing overlapping data components consistently occur in multiple realizations but locked to different time markers with variable inter-marker delays using an extended residue iteration decomposition (RIDE) algorithm. The present invention has applications in separating and analyzing event-related brain potential (ERP) data derived from single-trial responses.

If desired, the different functions discussed herein may be performed in a different order and/or concurrently with each other. Furthermore, if desired, one or more of the above-described functions may be optional or may be combined.

The embodiments disclosed herein may be implemented using general purpose or specialized computing devices, computer processors, or electronic circuitries including but not limited to digital signal processors (DSP), application specific integrated circuits (ASIC), field programmable gate arrays (FPGA), and other programmable logic devices configured or programmed according to the teachings of the present disclosure. Computer instructions or software codes running in the general purpose or specialized computing devices, computer processors, or programmable logic devices can readily be prepared by practitioners skilled in the software or electronic art based on the teachings of the present disclosure.

In some embodiments, the present invention includes computer storage media having computer instructions or software codes stored therein which can be used to program computers or microprocessors to perform any of the processes of the present invention. The storage media can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What is claimed is:

1. A method for separating and analyzing overlapping data components with variable delays in single trials comprising:
executing an initial latency estimation module for estimating a latency of an one or more unknown time-marker components of an one or more data components by a first template matching operation;
executing a first iterative module for decomposing the one or more data components by a minimization operation based on a known or the initially estimated latency of the one or more unknown time-marker components;
executing a second iterative module comprising:
further estimating a latency of the one or more decomposed data components without time-markers from the first iterative module wherein the latency is estimated by a second template matching operation between the one or more data components and single trials after removal of all other data components and the further estimated latency is applied to the first iterative module to further decompose the one or more data components;
applying a de-trend module to remove the trend noise in the decomposed one or more data components to prevent distortion in all the iterations other than the final iteration; and
applying a windowing module to refine the one or more data components using window functions in all the iterations other than the final iteration;
executing an iteration termination module to terminate the iteration between the second and the first iterative modules;
executing a baseline adjustment module to adjust the baseline of the separated one or more data components from the final iteration of the second iterative module; and
executing a reconstruction module to reconstruct the most probable representation of the added-up data components by summation of each separated component at their most probable latency across single trials.

2. The method according to claim 1, wherein the initial latency estimation module comprising one or more signal processing techniques including Woody's method.

3. The method according to claim 1, wherein the initial latency estimation module comprising one or more signal processing techniques including peak-picking.

4. The method according to claim 1, wherein the initial latency estimation module comprising one or more signal processing techniques including likelihood method.

5. The method according to claim 1, wherein the initial latency estimation module comprising one or more signal processing techniques including template matching using pre-defined templates.

6. The method according to claim 1, wherein the minimization operation comprising a Ln-norm operation.

7. The method according to claim 1, wherein the second template matching operation comprising a peak lag detection from cross-correlation between one data component and single trials wherein all other data components are removed.

8. The method according to claim 1, wherein the iteration termination module comprising an operation to terminate the iteration which further comprising a constraint of the estimated latency of the one or more components with unknown time-markers for each single trial to be monotonic.

9. The method according to claim 1, wherein the reconstruction module comprising an operation to reconstruct the most probable added-up data components by summation of all the decomposed data components respectively being located at their most probable latency across single trials.

10. The method according to claim 1, further comprising a module to provide estimates of the latency and amplitude information of the one or more data components in each single trial.

11. The method according to claim 1, wherein the reconstruction module comprising operations to obtain the waveforms for the one or more data components and the topographies at each time-marker of the one or more data components.

12. The method according to claim 1, further comprising a module to separate more than one data components with unknown time-marker components.

13. The method according to claim 12, wherein the separation module comprising the application of the initial latency estimation module in different time windows.

14. The method according to claim 1, wherein the one or more data components are event-related potential recordings.

15. The method according to claim 14, wherein the event-related potential recordings are recordings of brain activities.

16. The method according to claim 1, wherein the one or more data components are electroencephalography signal recordings.

17. The method according to claim 16, wherein the electroencephalography signal recordings are recordings of brain activities.
